(19) United States
(12) Patent Application Publication
(10) Pub. No.: US 2025/0072960 A1
(43) Pub. Date: Mar. 6, 2025

(54) DIELECTRIC COATING FOR CARDIAC ABLATION DEVICE

(71) Applicant: Boston Scientific Scimed, Inc., Maple Grove, MN (US)

(72) Inventors: Patrick Willoughby, Shoreview, MN (US); Emily Vance, Minneapolis, MN (US); Trey Henry Achterhoff, Arden Hills, MN (US); Thomas Grant, Minneapolis, MN (US); Scott Andrew McCullough, Plymouth, MN (US)

(21) Appl. No.: 18/816,787

(22) Filed: Aug. 27, 2024

Related U.S. Application Data
(60) Provisional application No. 63/535,966, filed on Aug. 31, 2023.

(51) Int. Cl. A61B 18/14; A61B 17/00; A61B 17/12; A61B 18/00

(52) U.S. Cl. CPC A61B 18/1492; A61B 17/1204; A61B 17/12122; A61B 17/12172; A61B 2017/00477; A61B 2017/1205; A61B 2018/00071; A61B 2018/00083; A61B 2018/00107; A61B 2018/00172; A61B 2018/00267; A61B 2018/00351; A61B 2018/00577; A61B 2018/00613

(57) ABSTRACT

A medical device includes an elongate hollow shaft having a shaft proximal end, a shaft distal end, and a lumen extending along the elongate hollow shaft. A handle is disposed at the shaft proximal end. A connector assembly having a connector assembly proximal end and a connector assembly distal end, the connector assembly proximal and distal ends being opposite ends of the connector assembly. An expandable frame is detachably disposed at the connector assembly distal end, the expandable frame including a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame.

### CROSS REFERENCE TO RELATED APPLICATIONS

This application claims priority to U.S. Provisional Patent Application No. 63/535,966 entitled “DIELECTRIC COATING FOR CARDIAC ABLATION DEVICE,” filed Aug. 31, 2023, which is herein incorporated by reference in its entirety.

### TECHNICAL FIELD

The present disclosure generally relates to devices, systems, and methods for ablating and occluding a body lumen or cavity and, more particularly, to devices, systems, and methods for occluding the left atrial appendage of the heart and ablation of tissue by electroporation.

### BACKGROUND

Atrial fibrillation (AF) is a common sustained cardiac arrhythmia affecting people worldwide. Serious consequences may come to those affected by AF. AF is the irregular, chaotic beating of the upper chambers of the heart where electrical impulses discharge so rapidly that the atrial muscle quivers or fibrillates. Episodes of AF may last a few minutes or several days. A serious consequence of AF is ischemic stroke. Most AF patients, regardless of the severity of their symptoms or frequency of episodes, require treatment to reduce the risk of stroke.

In patients with AF, blood tends to pool and form clots in an area of the heart called the left atrial appendage (LAA). The LAA is a pouch-like extension located in the upper left chamber of the heart. A blood clot that breaks loose from this area may migrate through the blood vessels and eventually plug a smaller vessel in the brain or heart resulting in a stroke or heart attack. It is known that a majority of blood clots in patients with AF are found in the LAA.

Treatment of AF may include surgically closing the LAA, epicardial LAA ligation, or delivering a device or mechanism across or into the LAA in order to occlude it. Occlusion devices for addressing AF typically utilize a metallic “cage” and/or fabric graft, which, when deployed, form a circular shape across and/or within the LAA. They are delivered to the treatment site via a catheter system.

In FIG. 1, a cross-sectional view of the human heart is shown. FIG. 1 also depicts a common technique whereby a catheter is threaded through the vasculature and into the heart to deliver an occlusion device to the LAA. Ideally, when the device is properly positioned within the LAA the occlusion device forms a seal with the wall of the LAA in order to prevent emboli or blood clots from passing back into the blood stream. Many known occlusion devices, however, are equipped with expandable frames that while sufficient to support a filter or membrane, have insufficient circumferential and/or radial strength to resist the distortive forces that the LAA exert on the occlusion device. As a result, the seal such devices form with the interior wall of the LAA is compromised as the expandable frame is bent into a more elliptical shape by the LAA. As a consequence, such devices may allow some material to exit the LAA and re-enter the blood stream.

Unexpected pericardial adhesions can delay or prevent successful occlusion operations. Known in the art are self-expanding nitinol frame structures with fixation barbs and a permeable polyterephthalate membrane that covers the atrial surface. These occlusion devices can be useful in hybrid ablation procedures. For examples, implantation of occlusion devices in a hybrid AF ablation setting (i.e., combination of thoracoscopic epicardial surgical and endocardial catheter ablation) can be a reliable option in cases where surgical LAA occlusion methods cannot be applied.

### SUMMARY

Example 1 is a medical device comprising an elongate hollow shaft having a shaft proximal end, a shaft distal end, and a lumen extending along the elongate hollow shaft; a handle disposed at the shaft proximal end; a connector assembly having a connector assembly proximal end and a connector assembly distal end, the connector assembly proximal and distal ends being opposite ends of the connector assembly; and an expandable frame that is detachably disposed at the connector assembly distal end, the expandable frame comprising a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame. The connector assembly is operatively connected to the expandable frame and configured to facilitate moving the expandable frame between a plurality of deployment positions.

Example 2 is the medical device of Example 1, wherein the expandable frame comprises a threaded socket and the connector assembly comprises a threaded wire that is receivable within the threaded socket such that the expandable frame is attached by threading the connector assembly into the expandable frame and is detached by unthreading the connector assembly from the expandable frame.

In Example 3, in the medical device of Examples 1 and 2, the connector assembly includes a hypotube that extends through the handles and a threaded wire that is attached to the hypotube and the expandable frame.

In Example 4, in the medical device of any of Examples 1-3, the handle includes a housing and a deployment assembly arranged together with the housing such that actuating the handle thereby moves the expandable frame between the plurality of deployment positions.

In Example 5, in medical device of Example 4, the deployment assembly includes a switch and a translator that is operatively connected to both the switch and the connector assembly such that actuating the handle comprises actuating the switch, the translator facilitating movement of the connector assembly relative to the elongate hollow shaft to thereby move the expandable frame between the plurality of deployment positions.

In Example 6, in the medical device of any of Examples 4 and 5, the deployment assembly comprises a torque knob with which to detach the expandable frame from the ablation catheter.

In Example 7, in the medical device of any of Examples 1-6, a biocompatible covering disposed over at least a part of the expandable frame.

In Example 8, in the medical device of any of Examples 1-7, the expandable frame comprises an electrode that facilitates delivering ablative energy to generate a volume of ablated tissue having a shape and size that corresponds to a deployment position of the expandable frame.

In Example 9, in the medical device of any of Examples 1-8, the expandable frame is formed as a closed basket with an ablation electrode positioned at a distal end of the expandable frame.

In Example 10, The medical device of any of Examples 1-9, the first portion of the expandable frame includes a proximal end to a proximal border.

In Example 11, in the medical device of Example 10, the second portion of the expandable frame extends from the proximal border to a distal end of the expandable frame.

In Example 12, in the medical device of Example 10, the second portion of the expandable frame extends from the proximal border to a distal border proximal from the distal end.

In Example 13, in the medical device of Example 12, the expandable frame includes a third portion coated with insulation, wherein the third portion extends from distal border to the distal end.

In Example 14, in the medical device of Example 13, the second portion extends between the proximal ring of anchors on the expandable frame and a distal ring of anchors on the expandable frame.

In Example 15, in the medical device of any of Examples 1-14, the interconnected members are axially surrounded by the dielectric layer in the first portion.

Example 16 is a medical device comprising: an elongate hollow shaft having a shaft proximal end, a shaft distal end, and a lumen extending along the elongate hollow shaft; a handle disposed at the shaft proximal end; a connector assembly having a connector assembly proximal end and a connector assembly distal end, the connector assembly proximal and distal ends being opposite ends of the connector assembly; and an expandable frame that is detachably disposed at the connector assembly distal end, the expandable frame comprising a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame. The connector assembly is operatively connected to the expandable frame and configured to facilitate moving the expandable frame between a plurality of deployment positions.

In Example 17, in the medical device of Example 16, the expandable frame comprises a threaded socket and the connector assembly comprises a threaded wire that is receivable within the threaded socket such that the expandable frame is attached by threading the connector assembly into the expandable frame and is detached by unthreading the connector assembly from the expandable frame.

In Example 18, in the medical device of Example 16, the connector assembly includes a hypotube that extends through the handles and a threaded wire that is attached to the hypotube and the expandable frame.

In Example 19, in the medical device of Example 16, the handle includes a housing and a deployment assembly arranged together with the housing such that actuating the handle thereby moves the expandable frame between the plurality of deployment positions.

In Example 20, in the medical device of Example 16, wherein a biocompatible covering disposed over at least a part of the expandable frame.

In Example 21, in the medical device of Example 16, the expandable frame comprises an electrode that facilitates delivering ablative energy to generate a volume of ablated tissue having a shape and size that corresponds to a deployment position of the expandable frame.

In Example 22, in the medical device of Example 16, the expandable frame is formed as a closed basket with an ablation electrode positioned at a distal end of the expandable frame.

In Example 23, in the medical device of Example 16, the first portion of the expandable frame includes a proximal end to a proximal border.

In Example 24, in the medical device of Example 23 the second portion of the expandable frame extends from the proximal border to a distal end of the expandable frame.

In Example 25, in the medical device of Example 23, the second portion of the expandable frame extends from the proximal border to a distal border proximal from the distal end.

In Example 26, in the medical device of Example 16, the expandable frame includes a third portion coated with insulation, wherein the third portion extends from distal border to the distal end.

In Example 27, in the medical device of claim 16, wherein the second portion extends between the proximal ring of anchors on the expandable frame and a distal ring of anchors on the expandable frame.

In Example 28, in the medical device of Example 16, wherein the interconnected members are axially surrounded by the dielectric layer in the first portion.

Example 29, is a method for occluding portions of a heart, the method comprising delivering an ablation catheter into the heart such that an intracardial portion of the ablation catheter is positioned adjacent a portion of the heart that is to be ablated The ablation catheter comprises an elongate hollow shaft having a shaft proximal end, a shaft distal end, and a lumen extending along the elongate hollow shaft; an expandable frame that is detachably disposed at the shaft distal end, the expandable frame comprising a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame. The ablation catheter also comprises a handle that is disposed at the shaft proximal end and operatively connected to the expandable frame such that actuation of the handle moves the expandable frame between the plurality of deployment positions. The ablation catheter is configured to direct energy to the expandable frame so as to ablate tissue at or around the second portion of the expandable frame; and deploys the expandable frame into the portion of the heart that is to be ablated.

In Example 30, the method of Example 29 further comprises positioning a deployed portion of the expandable frame to be adjacent tissue that is to be ablated, and generating ablative energy at the expandable frame to ablate tissue at or around the second portion of the expandable frame.

In Example 31, the method of Example 30 further comprises moving the expandable frame into at least one of the focal arrangement and the wide area arrangement.

In Example 32, in the method of Example 31, the ablation catheter further includes a connector assembly that extends from the handle to the expandable frame so as to operatively connect the handle to the expandable frame; and a deployment assembly that is arranged at the handle to correspond actuating the handle with directing the expandable frame to move between the plurality of deployment positions. The deployment assembly includes a switch and a translator that is operatively connected to both the switch and the connector assembly such that actuating the handle comprises actuating the switch, the translator facilitating movement of connector assembly relative to the elongate hollow shaft to thereby move the expandable frame between the plurality of deployment positions; and moving the expandable frame into at least one of the focal arrangement and the wide area arrangement comprises actuating the handle.

In Example 33, the method of Example 30 deploys the expandable frame into a portion of the heart that is to be ablated comprises detaching the expandable frame into a portion of the heart that is to be occluded.

In Example 34, in the method of Example 29, the expandable frame includes a third portion coated with insulation, wherein the third portion extends from distal border to the distal end.

In Example 35, in the method of Example 29, the interconnected members are axially surrounded by the dielectric layer in the first portion.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a cross-sectional illustration of the human heart depicting the left atrial appendage (LAA) and a mode of access by a catheter assembly through which one or more embodiments of the present disclosure may be deployed;

FIG. 2A is a plan view of an ablation catheter, according to principles of the present disclosure;

FIG. 2B is a cutaway view of the handle in the ablation catheter of FIG. 2A, according to principles of the present disclosure;

FIG. 3 is a perspective view of a handle for an ablation catheter, according to principles of the present disclosure;

FIG. 4 is an isolated perspective view of a switch and a translator included in the handle shown in FIG. 3;

FIG. 5 is a plan view of an expandable frame with double inverted proximal and distal hubs, according to principles of the present disclosure;

FIG. 6 is a plan view of an expandable frame with an inverted proximal hub and an external distal hub, according to principles of the present disclosure;

FIG. 7 is a plan view of the expandable frame in FIG. 5 with a biocompatible cover;

FIG. 8 is a plan view of the expandable frame in FIG. 6 with a biocompatible cover;

FIG. 9 is a flowchart of a method for performing a hybrid ablation procedure, according to principles of the present disclosure.

FIG. 10A is a plan view of an embodiment of an expandable frame with an electrically insulated portion and an exposed conductive portion; and

FIG. 10B is a plan view of another embodiment of an expandable frame with electrically insulated portions and an exposed conductive portion.

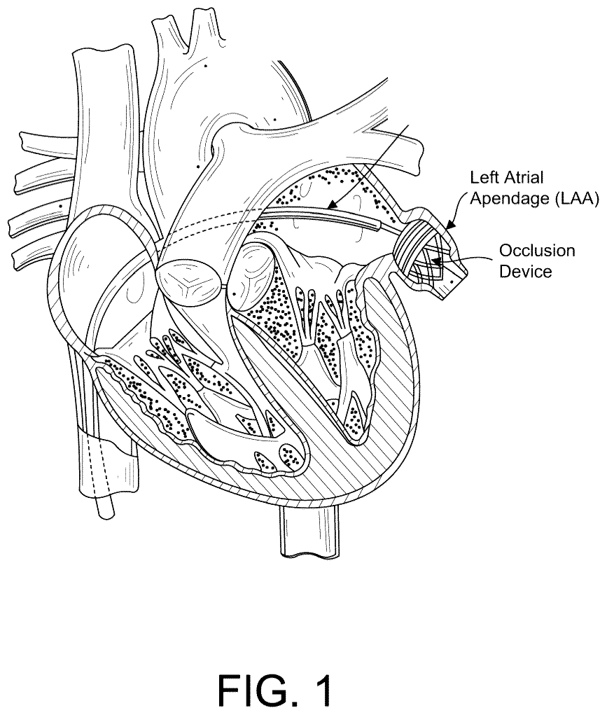

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

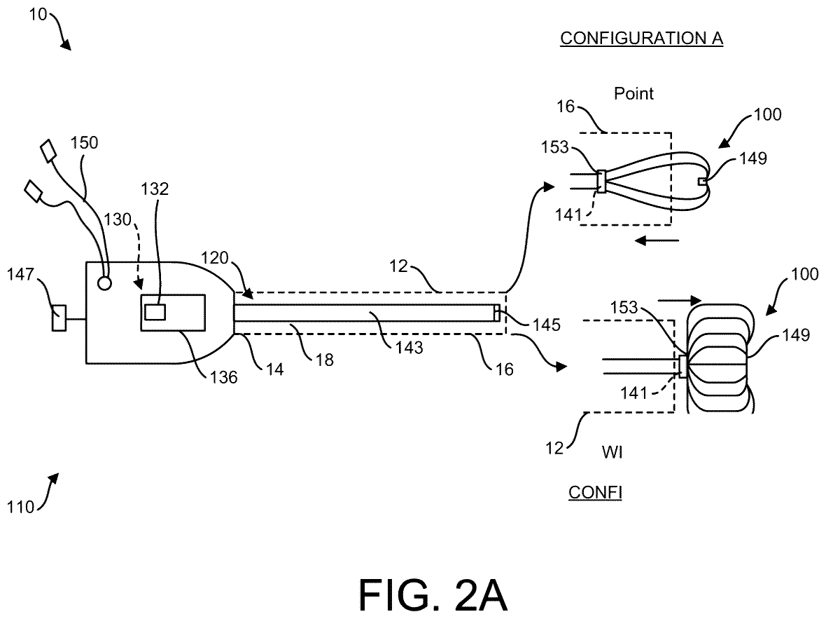

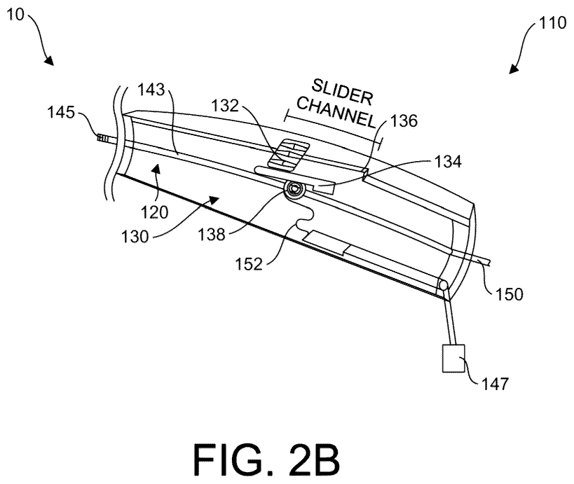

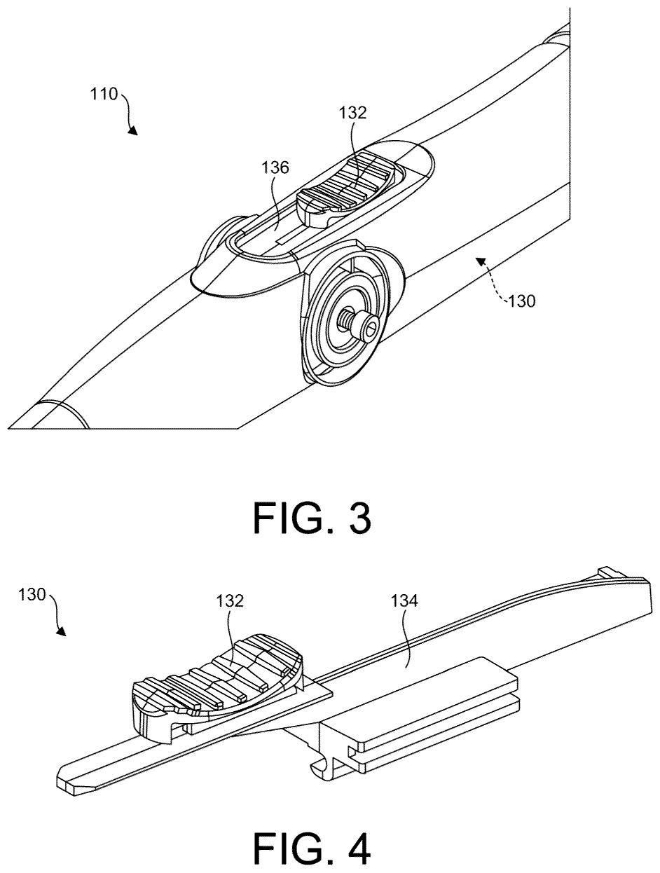

For purposes of this disclosure, like reference numerals in the figures shall refer to like features unless otherwise indicated. For example, reference numeral 100 refers to an expandable frame in FIG. 2A and also refers to an expandable frame in FIGS. 3 and 4.

Generally, disclosed herein are devices, systems, and methods for treatment of heart conditions related to fibrillations such as left atrial appendage fibrillation (LAAF). In this regard, examples of the present disclosure include occlusive implant delivery systems. These systems can perform hybrid ablation procedures using a single device. Such systems can include an ablation catheter as shown herein such as those with a detachable ablative, occlusion device (e.g., an occlusive implant) with an expandable frame that can be manipulated during operation to perform hybrid ablation procedures using different forms of ablation depending on the operation. For instance, the device can assume several arrangements, including a focal arrangement (see configuration A in FIG. 2A) and an expanded arrangement (e.g., a wide-shot arrangement at configuration B in FIG. 2A) that corresponds to a deployment position of the occlusion device. At least one of these arrangements can use one or more ablation techniques, such as radiofrequency (RF) ablation and pulsed field ablation (PFA). In this regard, the system also includes a generator (not shown) that is operatively connected to the ablation catheter 10 to cause the ablation catheter 10 to generate ablative energy at and/or around the device. These generators can be similar to those known in the art, such as those configured to perform PFA and RF ablation. These and more examples of principles of the present disclosure are discussed below.

In one or more aspects of the present disclosure, an occlusion device may include an expandable frame and a biocompatible covering disposed over at least a part of the expandable frame. Such an occlusion device of the present disclosure may be used to, for example, occlude the left atrial appendage (LAA) of the heart for the treatment of, for example, sustained cardiac arrhythmia (e.g., atrial fibrillation). When an occlusion device of the present disclosure is properly positioned within the LAA, the occlusion device may have sufficient circumferential and/or radial strength to form a seal with the wall of the LAA (and resist the distortive forces that the LAA may exert on the occlusion device) in order to, for example, prevent emboli or blood clots from passing back into the blood stream.

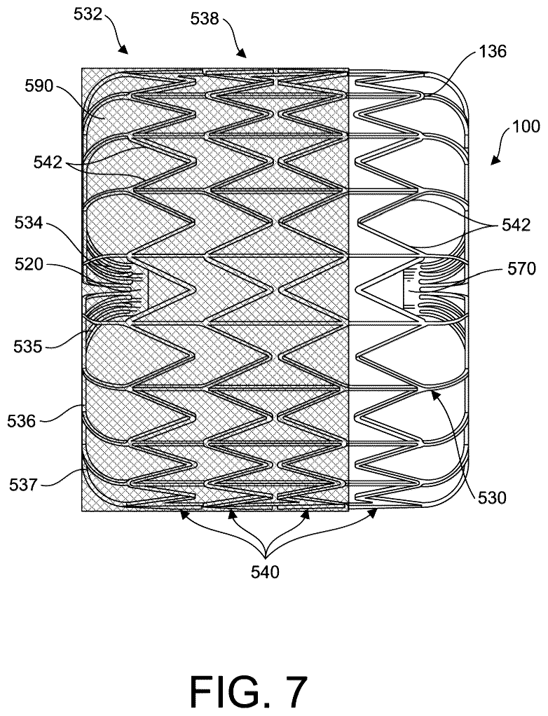

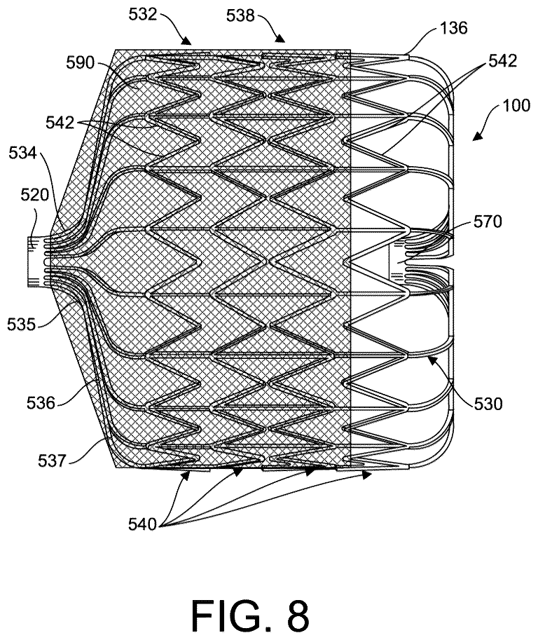

More detail about the ablation catheter 10 will be discussed with reference to FIGS. 2A and 2B. As shown here, the ablation catheter 10 includes an elongate hollow shaft 12 having a shaft proximal end 14, a shaft distal end 16, and a lumen 18 extending along the elongate hollow shaft 12; an expandable frame 100 that is detachably disposed at the shaft distal end 16; and a handle 110 that is disposed at the shaft proximal end 14. The expandable frame 100 has a plurality of deployment positions, which can include a first deployed position in which the expandable frame 100 is in a focal (e.g., narrow) arrangement (e.g., configuration A) and a second deployed position in which the expandable frame 100 is in a wide area arrangement (e.g., configuration B). Other deployment positions (such as those between or just beyond configurations A and B) are also contemplated and within the scope of this disclosure. As discussed further below, in examples, the ablation catheter 10 is configured to direct energy to the expandable frame 100 so as to ablate tissue at or around the expandable frame 100. Optionally, when the expandable frame 100 is formed as an occlusion device (see, e.g., FIGS. 7 and 8), the ablation catheter 10 is further configured to deploy the occlusion device by detaching it from the ablation catheter 10 as discussed in more detail below.

A connector assembly 120 operatively connects the expandable frame 100 to the handle 110. In examples, the connector assembly 120 extends from the handle 110 to the expandable frame 100 to thereby operatively connect the handle 110 to the expandable frame 100. The expandable frame 100 is disposed at a distal end of the connector assembly 120, and the handle 110 is disposed at a proximal end of the connector assembly 120. As discussed below, the connector assembly 120 can include one or more connector components (e.g., tubes, wires, or other connectors) that are interconnected. Under these circumstances, the connector assembly 120 can perform various functions, such as assisting with a deployment operation of the ablation catheter 10 as further discussed below. Of course, the one or more connector components can be integrally formed.

With reference to FIGS. 2A, 2B, 3, and 4, operation of the ablation catheter 10 can be facilitated via a deployment assembly 130 that is included with the handle 110. More specifically, as shown in the illustrated example in FIGS. 2A and 2B, the handle 110 includes a housing with the deployment assembly 130 arranged therein. The deployment assembly 130 includes a switch 132 and a translator 134 that is operatively connected to both the switch 132 and the connector assembly 120. The switch 132 is disposed within a corresponding slot 136 in the housing. Illustratively, the translator 134 is formed as an extension of the switch 132 that is connected to the connector assembly 120 via a bearing 138. In this regard, actuating the handle 110 may include actuating the switch 132 (e.g., proximally and/or distally relative to the housing). The translator 134 facilitates movement of the connector assembly 120 relative to the elongate hollow shaft 12 to thereby move the expandable frame 100 between the plurality of deployment positions. In this regard, actuating the handle 110 moves the expandable frame 100 between the plurality of deployment positions.

To facilitate deployment of the expandable frame 100 when it is an occlusion device, the deployment assembly 130 can be manipulated. For instance, with continued reference to FIGS. 2A and 2B, the expandable frame 100 may include a threaded socket 141 and the connector assembly 120 may include a connector 143 with a threaded portion 145 (e.g., at a distal end of the connector 143). As such, this connector 143 is receivable within the threaded socket 141 such that the expandable frame 100 is attached by threading the connector 143 (e.g., using the threaded portion 145) into the expandable frame 100 and is detached by unthreading the connector 143 from the expandable frame 100. The deployment assembly 130 may include a torque knob 147 with which to detach the expandable frame 100 from the ablation catheter 10. In examples, the connector 143 is formed at least partially as a hypotube that extends through the handle 110 and a threaded connector 143 or threaded portion 145 that is attached to the hypotube and the expandable frame 100 so as to be coupled between the hypotube and the expandable frame 100.

As discussed above, the ablation catheter 10 can generate electrical energy in the expandable frame 100 for ablation procedures. In this regard, illustratively, the expandable frame 100 is made of plurality of interconnected members formed of an electrically conductive material. Thus, in various embodiments, the entirety of the expandable frame is capable of functioning as an ablation electrode.

In various embodiments, the expandable frame includes an electrode 149 (e.g., an ablation electrode 149). The generator is configured to facilitate delivering ablative energy to generate a volume of ablated tissue having a shape and size that corresponds to the deployment position of the expandable frame 100. The expandable frame 100 is formed as a closed basket with an ablation electrode 149 positioned at a distal end of the expandable frame 100. In an ablation system, the ablation catheter 10 can be connected to a generator via a coupler 150 at the proximal end of the handle 110. Together, the ablation catheter 10 and generator are configured to generate energy to perform pulsed field ablation, for example. A flex connector 152 can ensure that the coupler 150 is in communication with the expandable frame 100 (e.g., via the connector 143 and/or wires extending from the expandable frame 100 to the flex connector 152) even during actuation of the connector assembly 120 (e.g., via movement of the switch 132 and/or translator 134).

Multiple electrodes can be disposed in the ablation catheter 10 to perform multipolar ablation. In examples, the electrode 149 can be a subcomponent of an electrode assembly. In this regard, the electrode assembly can include first and second ablation electrodes 149, 153. Illustratively, one of the first and second electrodes 149, 153 is disposed at a distal end of the expandable frame 100 and the other is disposed proximal to the ablation electrode 149 (e.g., on the expandable frame 100, the elongate hollow shaft 12, or the connector 143). Of course, other arrangements of electrodes in the electrode assembly are also contemplated. For instance, there are examples where the ablation electrode is disposed at the proximal end of the expandable frame 100.

Certain design considerations are useful when constructing an expandable frame for use during operation, for instance during a cryoablation procedure. Visualization in an electroanatomical mapping system can be performed via a navigation sensor integration as are known in the art. Some implementations can use fluoroscopy and TEE/ICE for visualization. Further, the expandable frame can be constructed from rigid materials that are navigable through body lumens and do not have any negative effects on long term LAAO.

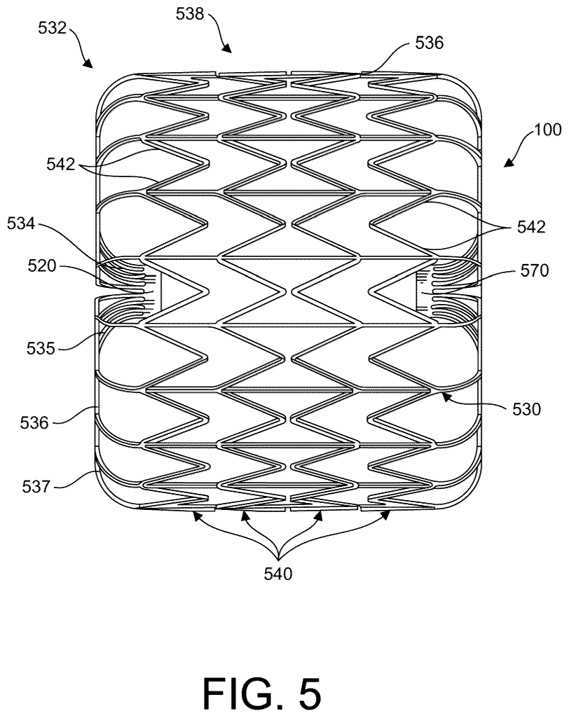

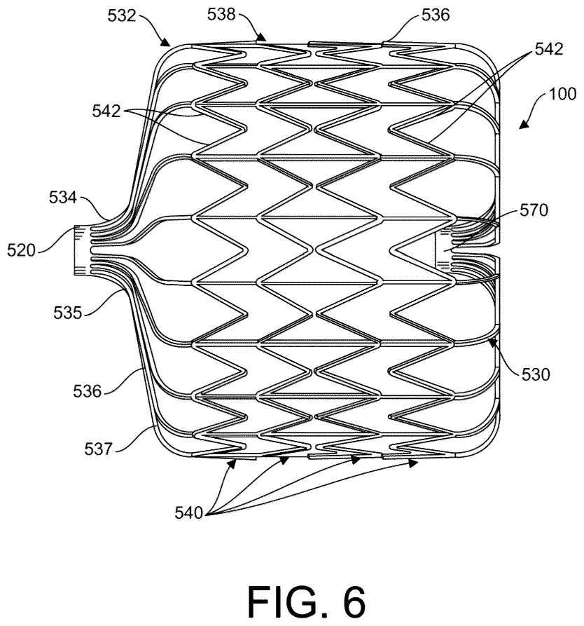

More details about the expandable frame 100 will now be discussed with reference to FIGS. 5-8. In particular, FIG. 5 is a side view of one or more embodiments of an expandable frame 100 of the present disclosure having closed proximal and distal ends. Illustratively, the ends are defined by an inverted proximal hub and an inverted distal hub. FIG. 6 is a side view of one or more embodiments of an expandable frame 100 of the present disclosure having closed proximal and distal ends. Illustratively, the ends are defined by an external proximal hub and an inverted distal hub (or vice versa). FIGS. 7 and 8 are similar to FIGS. 5 and 6 respectively except that the expandable frame 100 is implantable as an occlusive device.

As shown, an occlusion device may include an expandable frame 100 formed from, for example, a sheet. The expandable frame 100 may be suitable for use as a component of an occlusion device, which may also include covering 590 (e.g., a filter graft, membrane, etc.). Such a covering 590 may be supported by the expandable frame 100 (e.g., the covering 590 may extend over and from the proximal end of the expandable frame 100 toward the distal end of the expandable frame 100). The occlusion device (including the expandable frame 100 and covering 590) may include other components and may be combined with a delivery system for delivering the occlusion device to the LAA or other body lumen. In the one or more examples, the expandable frame 100 is depicted after manufacture, but before being loaded onto a catheter or deployed.

In the present disclosure, a beam 530 of expandable frame 100 may include a number of segments. For example, each beam 530 may include a first segment 532 extending from the first hub 520 to the first circumferentially extending column 540 of strut pairs 542 and a second segment 538 extending from the first circumferentially extending column 540 of strut pairs 542 to another (e.g., a second, third, fourth, etc.) circumferentially extending column 540 of strut pairs 542.

Illustratively, the expandable frame 100 includes a first hub 520 (e.g., a proximal cap or ring) from which a plurality of beams 530 (e.g., support beams 530) extend longitudinally therefrom. The first segment 532 (e.g., the proximal portion) of each beam 530 may also be considered as a radial component of the beam 530 because when the expandable frame 100 is expanded the predominant length of the first segment 532 may extend radially outward from first hub 520. When collapsed, the expandable frame 100 can be substantially flat or significantly compacted relative to the expanded state.

As shown in FIGS. 5-7, the first segment 532 of the beam 530 may include a first longitudinally extending region 534 that is immediately adjacent to the first hub 520. The first longitudinal extending region 534 may transition to the radially extending region 536 at interior curve 535. In one or more examples, the radially extending region 536 may then transition or turn back to the longitudinal direction at exterior curve 537. One or more examples of the present disclosure may include an expandable frame 100 including a plurality of beams 530 that terminate at a second hub 570 (e.g., a distal cap or ring). In at least one example the distal end of the expandable frame 100 may include a second hub 570 (e.g., the end may be closed), such that the longitudinally extending beams 530 turn radially inward near the distal end of the device to a distal cap or ring. In examples, the expandable frame 100 can be a self-expanding frame while in other examples the expandable frame 100 is a mechanically expanding frame. These examples are just some of many examples disclosed herein.

In some examples, in a deployed or expanded state, the first hub 520 (e.g., proximal ring) may be longitudinally adjacent (external) to the entire length of the beams 530, such that the beams 530 extend longitudinally away from the first hub 520 in a single longitudinal (distal) direction. In one or more examples, the first hub 520 may be inverted (internal) such that the beams 530 initially extend in a first (proximal) longitudinal direction away from the first hub 520 and then as the beams 530 turn and extend radially outward the beams 530 curve back over the first hub 520 in the opposite (distal) longitudinal direction. In one or more examples where the device has a second hub 570 (e.g., a distal ring), the second hub 570 may be configured with an internal (see, e.g., FIGS. 5 and 7) or external configuration (see, e.g., FIGS. 6 and 8). The internal or external positioning of the first hub 520 and second hub 570 may be the same or different. That is, in one or more examples, at least one of the first and second hubs 520, 570 may be inverted. For example, the first hub 520 and second hub 570 may be internally positioned (inverted) such as in the example shown in FIGS. 5 and 7. One hub (for example, first hub 520) may be externally positioned while the other hub (for example, second hub 570) may be inverted, such as in the example depicted in FIGS. 6 and 8. At least a portion of the beams 530 may be hooked (e.g., be J-shaped or C-shaped).

In examples wherein both the proximal end and distal end are closed, such as in the manner described herein, the columns 540 of strut pairs 542 may have a uniform orientation (all peaks “point” in the same direction) or, as shown, have opposing orientations relative to one or more other columns 540. In still other examples, an expandable frame 100 can include closed ends, and also include engagement anchors in the form of protrusions, indents, or other features of the expandable framework 100. For example, the framework may include one or more radially extending anchors (e.g., engagement barbs or other features) for improved securement of the framework into the surrounding tissue (e.g., the interior wall of the LAA) when the device is deployed.

Examples of the present disclosure include an expandable framework 100 that includes a biocompatible covering 590 disposed over at least a part of the expandable frame 100. In one or more examples, the covering 590 may take any of a wide variety of forms known to one of skill in the art. For example, a covering 590 may include a graft and/or a membrane and may include one or more layers. In one or more examples, a membrane or other covering 590 may be disposed over and about most of the proximal end of expandable frame 100. For example, a covering 590 may be substantially bowl-shaped, with an opening that extends around the portion of the occlusion device having the greatest diameter in the second configuration. In the one or more examples that include anchors as discussed above, the anchors may penetrate the covering 590 in both the first and second configurations (e.g., unexpanded and expanded states) to secure the covering 590 on the expandable frame 100. The covering 590 may be any of a wide variety of biocompatible fabric, membrane, or material known to one of skill in the art. For example, the covering 590 may be constructed of one or more layers of polyethylene terephthalate (PET). It should be recognized that the coverings described herein may be suitable for use with any of the examples of the expandable frame 100 shown or described herein.

Other suitable covering 590 materials may be employed as well. Examples may include, but are not limited to, polyethylene, polypropylene, polyvinyl chloride, polytetrafluoroethylene, including expanded polytetrafluoroethylene (ePTFE), fluorinated ethylene propylene, polyvinyl acetate, polystyrene, poly(ethylene terephthalate), naphthalene, dicarboxylate derivatives, such as polyethylene naphthalate, polybutylene naphthalate, polytrimethylene naphthalate, and trimethylenediol naphthalate, polyurethane, polyurea, silicone rubbers, polyamides, polyimides, polycarbonates, polyaldehydes, polyether ether ketone, natural rubbers, polyester copolymers, styrene-butadiene copolymers, polyethers, such as fully or partially halogenated polyethers, and copolymers and combinations thereof.

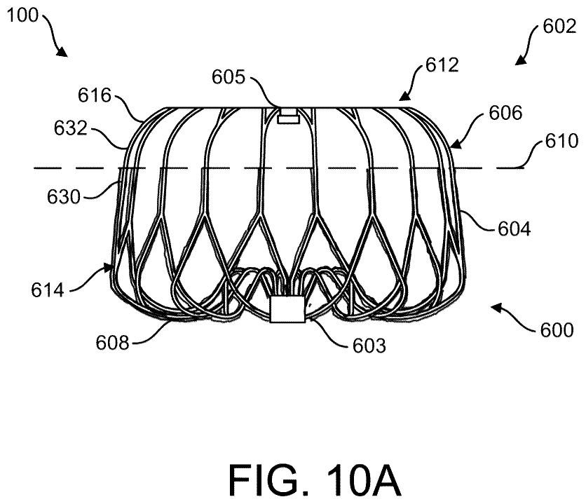

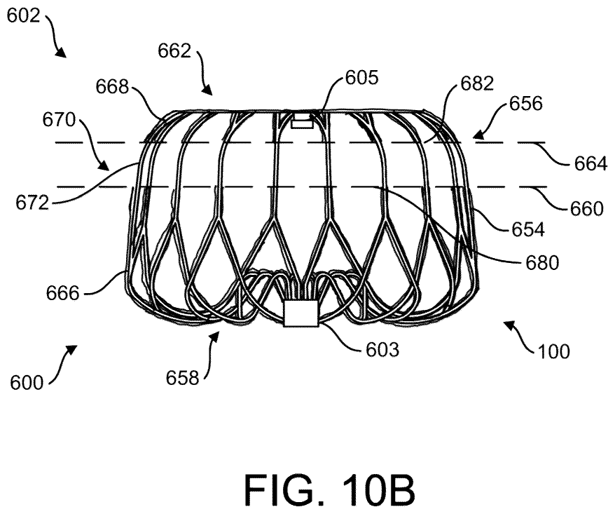

FIGS. 10A and 10B show an expandable frame 100 having an electrically insulative covering 600 disposed on conductive struts 602, such as nitinol struts, extending between a proximal threaded socket 603 to receive a connector assembly 603 to a distal puck 605. The delivery of PFA energy (or RF energy, as well) to a relatively large conductive frame, as well as a conductive frame with a relatively large distance between struts, can result in low impedance at the generator, which can result in relatively less favorable lesions in the myocardial tissue from ablation. The partially insulated expandable frames concentrate delivered energy to the exposed portions of the frame to achieve more favorable lesions. While an external hub configuration (see, e.g., FIG. 6), the concepts described apply similarly to an expandable frame having an internal hub configuration (see, e.g., FIG. 5). In various embodiments, portions of the expandable frame 100 are covered with an outer coating, thereby insulating a portion of the outer surface of the expandable frame 100. In other embodiments, a portion of the struts and columns are coated with an insulative material. In various embodiments, the insulative material is a dielectric material. In some embodiments, the partially insulated frame can further include a covering (such as that illustrated and described with respect to FIGS. 7-8). The application of the electrically insulative covering 600 at locations on the conductive struts 602 serves to reduce the exposed or bare conductive surface area of the frame acting as an electrode. The electrically insulative covering 600 concentrates ablation energy to the exposed portions of the of the frame 100.

FIG. 10A illustrates an embodiment having an expandable frame 100 including a proximal portion 604 extending from a proximal end 608 to a border 610 as a plane bisecting the frame 100 and a distal portion 606 extending from the border 610 to a distal end 612. The proximal portion 604 of the frame 100 includes electrically insulated struts 614 and a distal portion 606 with exposed conductive struts 616. Embodiments are contemplated including a distal portion of the expandable frame including insulated struts and a proximal portion having exposed struts. The frame 100 of the particular embodiment shown in FIG. 10A includes a substantial majority of the proximal portion 604 with struts 602 covered or coated with the insulative material 600. For example, the embodiment of FIG. 10A illustrates the border 610 distal to a ring of proximal anchors 630, such as hooks on the frame 100, and the border 610 near a ring of distal anchors 632, such as hooks on the frame 100 with the exposed struts 616 disposed around the frame 100 distal the proximal anchors 630 and the proximal the distal anchors 632.

FIG. 10B illustrates an embodiment having an expandable frame 100 including a proximal portion 654 extending from a proximal end 658 to a proximal border 660 as a plane extending through the frame 100, and a distal portion 656 extending from a distal end 662 to a distal border 664 as a plane extending through the frame 100. The proximal portion 654 and distal portion 656 include insulated struts 666, 668, respectively, i.e., the conductive struts 602 in the proximal and distal portions 654, 656, are covered in insulative material 600. The expandable frame 100 further includes a medial portion 670 extending from the proximal border 660 to the distal border 664 with electrically exposed conductive struts 672, i.e., the conductive struts 602 in the medial portion 670 are not covered with insulative material 600. Embodiments are contemplated having exposed distal and proximal portions with an electrically insulated medial portion. The frame 100 of the particular embodiment shown in FIG. 10B includes a substantial majority of the proximal and distal portions 654, 656 with struts 602 covered or coated with the insulative material 600. For example, the embodiment of FIG. 10B illustrates the proximal border 660 near a ring of proximal anchors 680, such as hooks on the frame 100, and the distal border 664 near a ring of distal anchors 682, such as hooks on the frame 100 with the medial portion 670 of exposed struts 672 disposed around the frame 100 between the proximal anchors 680 and the distal anchors 682.

The insulator 600 is an electrical insulator that resists delivery of electrical energy on the expandable frame 100 through the insulator 600. In some embodiments, the insulator 600 completely surrounds the conductive struts on which the insulator 600 is disposed. For example, the insulator 600 extends completely around the strut axial element in the embodiment and does not provide radial gaps of insulation to expose the conductor of the strut. In other embodiments, the insulator 600 extends around the strut axial element on the regions of the strut 602 that will contact tissue. In some embodiments, the exposed portions of the frame can include insulator on the axial portions of the struts that do not contact tissue, such as the inner frame facing an internal cavity. In some embodiments, the insulator 600 does not include longitudinal gaps to expose the conductor of the strut until reaching the insulation border 640, 642, 644 with exposed struts 614, 636. In some embodiments, the insulator 600 is constructed from a polymer applied to the frame 100 such as via a dip coating or spray coating the frame 100. In some embodiments, the insulator 600 is tubular polymer such as a shrink fit polymer disposed about the frame 100 and attached in place. In some embodiments, the insulator 600 is a metal oxide formed by a manufacturing process—such as physical vapor deposition or physical vapor transport (or type of vacuum deposition), sputtering, or an electrochemical process—to deposit a thin film or coating on the frame. Portions of the conductive struts that are intended to remain uncoated, or exposed, can be masked prior to coating, and the masking is removed to expose the uncoated conductive material of the finished frame. For example, the medial portion 670 of struts 672 can be masked prior to coating or covering the frame with insulative material, and the mask can later be removed to expose uncovered struts 672.

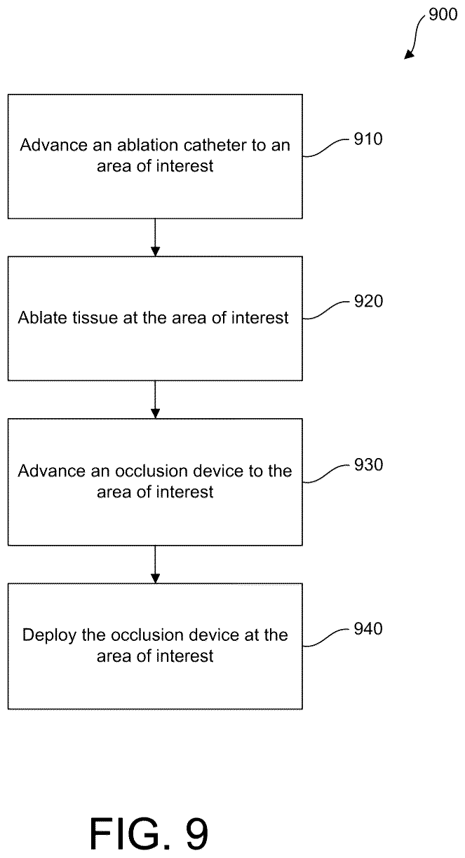

According to principles of the present disclosure, ablation and occlusion methods are also disclosed herein. Each of these methods can use ablation catheters similar to those disclosed elsewhere herein. For instance, as shown in FIG. 9, a method 900 for performing a hybrid ablation procedure and related methods are disclosed. At step 910, the method 900 can include advancing a distal end of the catheter through a patient's vasculature to an area of interest, such as into the heart to deliver an occlusion device to the LAA. In this regard, continuing with this heart example, the ablation catheter can be positioned adjacent heart tissue that is to be ablated. In one embodiment, the exposed or uncoated portion of the ablation catheter is positioned adjacent heart tissue that is to be ablated, and an insulated or coated portion of the ablation catheter is positioned adjacent to heart tissue that is not to be ablated. At step 920, the method 900 can include creating scar tissue inside the heart via one or more ablation techniques (e.g., RF and/or PFA ablation). When deploying an occlusion device is desirable, the method 900 can include advancing an occlusion device to the LAA at step 930 and deploying the device therein at step 940. When the device is properly positioned within the LAA, the occlusion device forms a seal with the wall of the LAA to prevent emboli and/or blood clots from passing back into the blood stream.

Using the ablation catheters disclosed elsewhere herein, the expandable frame employed in these methods can be used for ablation, occlusion, or both. For instance, as discussed above, an operator can have the option to use both narrow and wide-area ablation with the expandable frame. Ablation typically occurs before the expandable frame is deployed depending on the application. Transitioning between the narrow and expanded states of the expandable frame (or from one state to the other) can corresponded to actuating a connector assembly of the ablation catheter. In this regard, distal-to-proximal translation (e.g., of a switch) can move the expandable frame from a pre-expansion state within an elongate shaft of the ablation catheter to the narrow state just outside the elongate shaft, and further advancement in the same direction can move the expandable frame to the wide-area state further still outside the elongate shaft. Of course, there are examples where other movements (e.g., distal to proximal, rotations, etc.) of the connector assembly similarly advance the expandable frame. Deploying the device at step 940 can include detaching the occlusion device from the ablation catheter using a deployment assembly. In this regard, this step 940 can include unthreading the occlusion device from a connector that is in threaded engagement with the occlusion device. The connector can be in the form of a hypotube that is operatively connected to a torque knob at a proximal end of the ablation catheter for easy access such that rotating the torque knob rotates the connector to thereby thread/unthread the occlusion device. Other attachment/detachment methods, such as those known in the art and disclosed elsewhere herein, are also contemplated.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device comprising:
an elongate hollow shaft having a shaft proximal end, a shaft distal end, and a lumen extending along the elongate hollow shaft;
a handle disposed at the shaft proximal end;
a connector assembly having a connector assembly proximal end and a connector assembly distal end, the connector assembly proximal and distal ends being opposite ends of the connector assembly; and
an expandable frame that is detachably disposed at the connector assembly distal end, the expandable frame comprising a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame; and
wherein the connector assembly is operatively connected to the expandable frame and configured to facilitate moving the expandable frame between a plurality of deployment positions.

2. The medical device of claim 1, wherein the expandable frame comprises a threaded socket and the connector assembly comprises a threaded wire that is receivable within the threaded socket such that the expandable frame is attached by threading the connector assembly into the expandable frame and is detached by unthreading the connector assembly from the expandable frame.

3. The medical device of claim 1, wherein the connector assembly includes a hypotube that extends through the handles and a threaded wire that is attached to the hypotube and the expandable frame.

4. The medical device of claim 1, wherein the handle includes a housing and a deployment assembly arranged together with the housing such that actuating the handle thereby moves the expandable frame between the plurality of deployment positions.

5. The medical device of claim 1, wherein a biocompatible covering disposed over at least a part of the expandable frame.

6. The medical device of claim 1, wherein the expandable frame comprises an electrode that facilitates delivering ablative energy to generate a volume of ablated tissue having a shape and size that corresponds to a deployment position of the expandable frame.

7. The medical device of claim 1, wherein the expandable frame is formed as a closed basket with an ablation electrode positioned at a distal end of the expandable frame.

8. The medical device of claim 1, wherein the first portion of the expandable frame includes a proximal end to a proximal border.

9. The medical device of claim 8, wherein the second portion of the expandable frame extends from the proximal border to a distal end of the expandable frame.

10. The medical device of claim 8, wherein the second portion of the expandable frame extends from the proximal border to a distal border proximal from the distal end.

11. The medical device of claim 1, wherein the expandable frame includes a third portion coated with insulation, wherein the third portion extends from distal border to the distal end.

12. The medical device of claim 1, wherein the second portion extends between the proximal ring of anchors on the expandable frame and a distal ring of anchors on the expandable frame.

13. The medical device of claim 1, wherein the interconnected members are axially surrounded by the dielectric layer in the first portion.

14. A method for occluding portions of a heart, the method comprising:
delivering an ablation catheter into the heart such that an intracardial portion of the ablation catheter is positioned adjacent a portion of the heart that is to be ablated, the ablation catheter comprising:
an elongate hollow shaft having a shaft proximal end, a shaft distal end, and a lumen extending along the elongate hollow shaft;
an expandable frame that is detachably disposed at the shaft distal end, the expandable frame comprising a plurality of interconnected members formed of an electrically conductive material, wherein the interconnected members in a first portion of the expandable frame are coated with a dielectric layer, and wherein the interconnected members in a second portion of the expandable frame are uncoated and configured as an electrode region of the expandable frame; and
a handle that is disposed at the shaft proximal end and operatively connected to the expandable frame such that actuation of the handle moves the expandable frame between the plurality of deployment positions, and
wherein the ablation catheter is configured to direct energy to the expandable frame so as to ablate tissue at or around the second portion of the expandable frame; and
deploying the expandable frame into the portion of the heart that is to be ablated.

15. The method of claim 14, further comprising positioning a deployed portion of the expandable frame to be adjacent tissue that is to be ablated, and generating ablative energy at the expandable frame to ablate tissue at or around the second portion of the expandable frame.

16. The method of claim 15, further comprising moving the expandable frame into at least one of the focal arrangement and the wide area arrangement.

17. The method of claim 16, wherein the ablation catheter further includes:
a connector assembly that extends from the handle to the expandable frame so as to operatively connect the handle to the expandable frame; and
a deployment assembly that is arranged at the handle to correspond actuating the handle with directing the expandable frame to move between the plurality of deployment positions, the deployment assembly including a switch and a translator that is operatively connected to both the switch and the connector assembly such that actuating the handle comprises actuating the switch, the translator facilitating movement of connector assembly relative to the elongate hollow shaft to thereby move the expandable frame between the plurality of deployment positions; and
wherein moving the expandable frame into at least one of the focal arrangement and the wide area arrangement comprises actuating the handle.

18. The method of claim 15, wherein deploying the expandable frame into a portion of the heart that is to be ablated comprises detaching the expandable frame into a portion of the heart that is to be occluded.

19. The method of claim 14, wherein the expandable frame includes a third portion coated with insulation, wherein the third portion extends from distal border to the distal end.

20. The method of claim 14, wherein the interconnected members are axially surrounded by the dielectric layer in the first portion.
